(19) United States
(12) Patent Application Publication
(10) Pub. No.: US 2009/0240155 A1
(43) Pub. Date: Sep. 24, 2009

(54) SLEEP CONDITION MEASURING APPARATUS AND METHOD

(75) Inventors: Kanako Nakayama, Yokohama-shi (JP); Takuji Suzuki, Yokohama-shi (JP); Kenichi Kameyama, Kawasaki-shi (JP); Kazushige Ouchi, Saitama-shi (JP)

Correspondence Address: TUROCY & WATSON, LLP, 127 Public Square, 57th Floor, Key Tower, CLEVELAND, OH, 44114, US

(73) Assignee: KABUSHIKI KAISHA TOSHIBA, Tokyo (JP)

(21) Appl. No.: 12/407,351

(22) Filed: Mar. 19, 2009

(30) Foreign Application Priority Data
Mar. 21, 2008 (JP) 2008-074292

(51) Int. Cl. A61B 5/02; A61B 5/11

(52) U.S. Cl. 600/500; 600/595

(57) ABSTRACT

A sleep condition measuring apparatus includes a determination unit configured to determine a first timing indicating that the user has fallen asleep and a second timing indicating that the user has waken up, based on body motion of a user, an acquisition unit configured to acquire a first index indicating activity of a sympathetic nerve of the user and a second index indicating activity of a parasympathetic nerve of the user, based on a pulse wave interval of the user, a calculation unit configured to calculate a dominance level of the second index over the first index every predetermined time, and a calculation unit configured to calculate a third index indicating quality of sleep of the user from the first timing until the second timing, using the dominance level and a weight decreasing as time elapses from the first timing.

### CROSS-REFERENCE TO RELATED APPLICATIONS

This application is based upon and claims the benefit of priority from prior Japanese Patent Application No. 2008-074292, filed Mar. 21, 2008, the entire contents of which are incorporated herein by reference.

### BACKGROUND OF THE INVENTION

1. Field of the Invention

The present invention relates to a sleep condition measuring apparatus and a sleep condition measuring method which measure indices indicating the quality of a user's sleep.

2. Description of the Related Art

A user's sleep condition is conventionally comprehensively determined based on the user's biological information, called a polysomnogram. A polysomnogram includes a user's brain waves, respiration, myoelectric potential, and electrocardiography. However, polysomnographic measurement requires a large-scale measuring apparatus. Therefore, much attention has been paid to techniques for determining the user's sleep condition based on biological information such as a user's pulse wave interval and a user's body motion that can be acquired more easily than the polysomnogram.

A sleep condition determining method described in JP-A H07-143972 derives indices indicating the user's autonomic nerves from a frequency spectrum component of the user's pulse wave interval, to determine the sleep condition based on the indices. Specifically, the sleep condition determining method derives, as the index, power spectra LF and HF of a low frequency region (close to 0.05 to 0.15 Hz) and a high frequency region (close to 0.15 to 0.4 Hz) of the frequency spectrum component.

A system determining the sleep condition and nocturnal awakening described in JP-A 2002-34955 determines the depth of sleep based on the user's pulse wave interval and body motion. Specifically, the depth of sleep is classified into arousal, REM sleep, non-REM sleep, and nocturnal awakening, and a determination result for the depth of sleep during the user's sleep is displayed as time sequence data.

A sleep condition measuring apparatus described in JP-A 2007-130181 derives indices that are relatively easy to understand in general, such as the user's easiness-of-falling-asleep, sleep duration, sleep efficiency, comfort level, sleep rhythm index, body motion, and nocturnal awakening.

With the sleep condition determining method described in JP-A H07-143972 and the system determining the sleep condition and nocturnal awakening which system is described in JP-A 2002-34955, the time sequence data on the user's depth of sleep can be displayed as a determination result for the sleep condition. However, if the method or the system is used during the user's daily life, the user needs to determine the user's own sleep condition from the time sequence data on the depth of sleep. Thus, if the user's knowledge of sleep is insufficient, determining the user's own sleep condition from the determination result is difficult.

A sleep condition measuring apparatus described in JP-A 2007-130181 can present the user with indices that are relatively easy to understand, and is thus suitable for daily use. However, the comfort level, an index indicating the quality of the user's sleep, is determined by simply integrating the dominance level of the user's parasympathetic nerves over the user's sympathetic nerves. Thus, the comfort level may exhibit a larger value than required, depending on the active conditions of the user's parasympathetic and sympathetic nerves. That is, the comfort level may be calculated for a particular type of sleepless person.

### BRIEF SUMMARY OF THE INVENTION

According to an aspect of the invention, there is provided a sleep condition measuring apparatus comprising: a first detection unit configured to detect body motion of a user; a determination unit configured to determine a first timing indicating that the user has fallen asleep and a second timing indicating that the user has waken up, based on the body motion; a second detection unit configured to detect a pulse wave interval of the user; an acquisition unit configured to acquire a first index indicating activity of a sympathetic nerve of the user and a second index indicating activity of a parasympathetic nerve of the user, based on the pulse wave interval; a first calculation unit configured to calculate a dominance level of the second index over the first index every predetermined time; and a second calculation unit configured to calculate a third index indicating quality of sleep of the user from the first timing until the second timing, using the dominance level and a weight decreasing as time elapses from the first timing.

### BRIEF DESCRIPTION OF THE SEVERAL VIEWS OF THE DRAWING

FIG. 1 is a block diagram showing a sleep condition measuring apparatus according to a first embodiment;

FIG. 2 is a diagram showing an example of installation of the sleep condition measuring apparatus in FIG. 1 and a pulse wave sensor;

FIG. 3A is a graph showing an example of body motion data acquired by a body motion data processing unit in FIG. 1;

FIG. 3B is a graph showing a temporal variation in a derivative obtained by subjecting the body motion data in FIG. 3A to temporal differentiation;

FIG. 3C is a graph showing a variation in body motion data which is a square root of square summation of each derivative in FIG. 3B;

FIG. 4A is a diagram illustrating detection of pulse wave interval data by a pulse wave data processing unit in FIG. 1;

FIG. 4B is a graph showing pulse wave interval data obtained based on FIG. 4A;

FIG. 4C is a graph showing an example of interpolated pulse wave interval data obtained by interpolation of the pulse wave interval data in FIG. 4B performed by a pulse wave interval interpolating unit in FIG. 1;

FIG. 5A is a graph showing an example of pulse wave interval data analyzed by a pulse wave interval analyzing unit in FIG. 1;

FIG. 5B is a graph showing results of the analysis of the data in FIG. SA;

FIG. 6 is a diagram illustrating a relationship between an autonomic nerve index for a non-sleepless user acquired by an autonomic nerve index acquiring unit in FIG. 1 and the user's depth of sleep;

FIG. 7A is a graph showing an example of a temporal variation in the autonomic nerve index for a non-sleepless user A acquired by the autonomic nerve index acquiring unit in FIG. 1;

FIG. 7B is a graph showing an example of a temporal variation in the autonomic nerve index for a sleepless user B acquired by the autonomic nerve index acquiring unit in FIG. 1;

FIG. 7C is a graph showing an example of a temporal variation in the autonomic nerve index for a sleepless user C acquired by the autonomic nerve index acquiring unit in FIG. 1;

FIG. 7D is a graph showing an example of a temporal variation in the autonomic nerve index for a sleepless user D acquired by the autonomic nerve index acquiring unit in FIG. 1;

FIG. 8 is a diagram showing an example of the dominance level of the parasympathetic nerves calculated by a parasympathetic nerve dominance level calculating unit in FIG. 1;

FIG. 9 is a diagram illustrating calculation of a comfort level by a sleep condition measuring apparatus according to a second embodiment; and

FIG. 10 is a diagram illustrating effects of the sleep condition measuring apparatus according to the second embodiment.

### DETAILED DESCRIPTION OF THE INVENTION

Embodiments of the present invention will be described below with reference to the drawings.

### First Embodiment

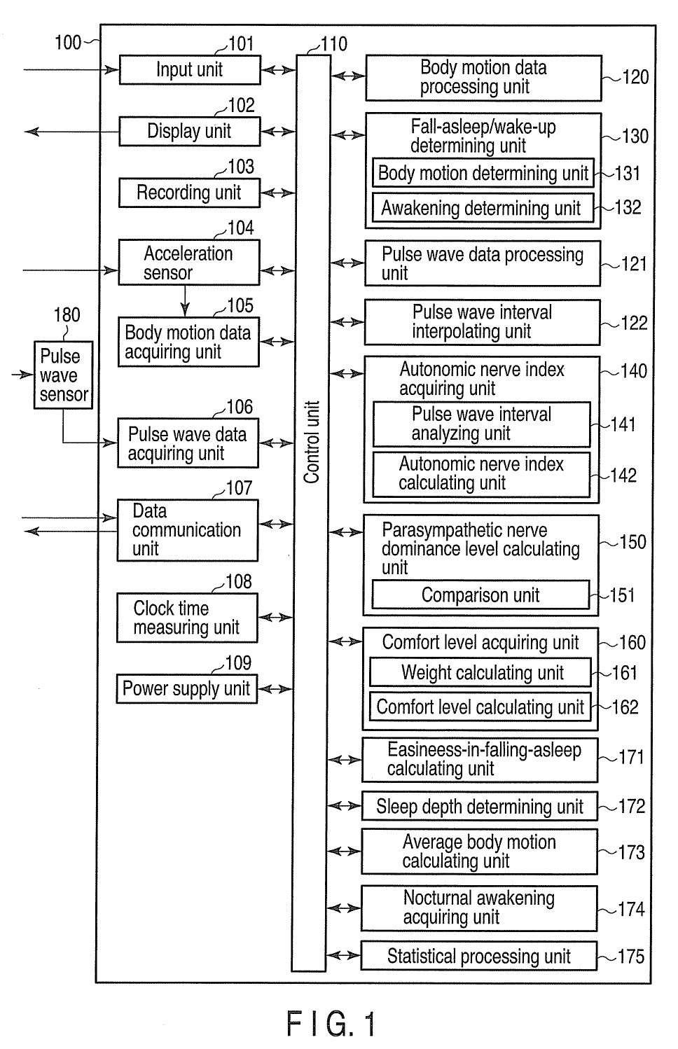

As shown in FIG. 1, a sleep condition measuring apparatus 100 according to a first embodiment of the present invention includes an input unit 101, a display unit 102, a recording unit 103, an acceleration sensor 104, a body motion acquiring unit 105, a pulse wave data acquiring unit 106, a data communication unit 107, a clock time measuring unit 108, a power supply unit 109, a control unit 110, a body motion data processing unit 120, a fall-asleep/wake-up determining unit 130, a pulse wave data processing unit 121, a pulse wave interval interpolating unit 122, an autonomic nerve index acquiring unit 140, a parasympathetic nerve dominance level calculating unit 150, a comfort level acquiring unit 160, an easiness-of-falling-asleep acquiring unit 171, a sleep depth determining unit 172, an average body motion amount calculating unit 173, a nocturnal awakening acquiring unit 174, and a statistical processing unit 175.

An external pulse wave sensor 180 is connected to the sleep condition measuring apparatus 100. The pulse wave sensor 180 is a photoplethysmographic sensor including a light emitting element (for example, a blue or green LED) with wavelengths in a blue or green band, and a light receiving element (for example, a photo diode). As described below, when the pulse wave sensor 180 is installed around a user's finger, a blue LED or the like irradiates the finger with light. The photo diode then catches a variation in reflected light resulting from a change in a blood flow in capillary vessels inside the finger, and converts the variation into a current. An output current from the photo diode is supplied to the pulse wave data acquiring unit 106.

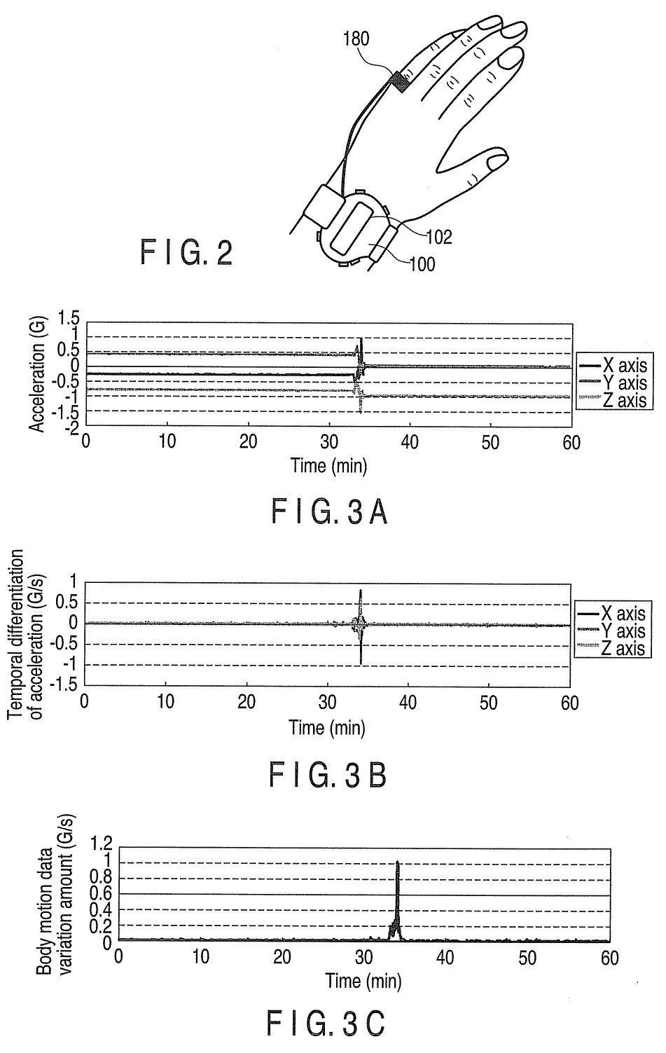

As shown in FIG. 2, for example, the sleep condition measuring apparatus 100 is shaped like a watch and installed around the user's wrist. The pulse wave sensor 180 is installed around the user's finger. In another example of installation of the sleep condition measuring apparatus 100 and the pulse wave sensor 180, the pulse wave sensor 180 may be installed around the user's hand or arm. Alternatively, the pulse wave sensor 180 may be provided inside the sleep condition measuring apparatus 100.

Input unit 101 receives and inputs user inputs from the user to the control unit 110. The user inputs include an instruction to turn on or off a power source for the sleep condition measuring apparatus 100, an instruction to activate or inactivate a sleep condition measuring function, and an instruction to change displayed content on the display unit 102, described below.

The display unit 102 is a display and/or a speaker which visually and/or auditorily indicates the various indices to the user as measurement results of the sleep condition. A display target of the display unit 102 is controlled by the control unit 110.

The following data is recorded in the recording unit 103 by the control unit 110: pulse wave data, body motion data, and the like which are acquired from the user and which will be described below, pulse wave interval data, and the amount of body motion. The various indices and threshold values used to derive these indices are also recorded in the recording unit 103 by the control unit 110.

The acceleration sensor 104 includes an accelerometer that measures acceleration in directions of three axes (x axis, y axis, and z axis), and is provided inside the sleep condition measuring apparatus 100. The acceleration sensor 104 measures the acceleration in the directions of the three axes of the sleep condition measuring apparatus 100, for example, every 50 msec. The acceleration sensor 104 then inputs measurement results, that is, data (for example, analog amounts) indicating the amounts of body motion in the directions of the three axes in the user's installation region.

The body motion data acquiring unit 105 adjusts the gain and offset of the data on the amount of body motion from the acceleration sensor 104. The body motion data acquiring unit 105 then performs an analog-to-digital conversion as required, to obtain the user's body motion data. The body motion data acquired by the body motion acquiring unit 105 is recorded in the recording unit 103 via the control unit 110. Specifically, the body motion acquiring unit 105 can be implemented by an adjustment circuit that adjusts the gain and offset and a 10-bit converter for the analog-to-digital conversion.

The pulse wave data acquiring unit 106 converts the output current from the pulse wave sensor 180 into a voltage and amplifies the voltage. The pulse wave data acquiring unit 106 then extracts a required frequency component and subjects the voltage from which the component has been extracted to an analog-to-digital conversion to obtain pulse wave data. The pulse wave data acquired by the pulse wave data acquiring unit 106 is recorded in the recording unit 103 via the control unit 110. Specifically, the pulse wave data acquiring unit 106 is implemented by a current-voltage converter, an amplifier, a filter, and a 10-bit A/D converter. The filter may be a combination of a high-pass filter (cutoff frequency: 0.1 Hz) and a low-pass filter (cutoff frequency: 50 Hz), or a band-pass filter.

In accordance with requests from the control unit 110, the data communication unit 107 transmits and receives various data to and from another sleep condition measuring apparatus, a personal computer, PDA, a cellular phone, or the like through a wired or wireless network.

The clock time measuring unit 108 is, for example, a timer for measuring clock time. For example, the clock time measuring unit 108 measures a duration from the time when the power source for the sleep condition measuring apparatus 100 is turned on or a duration from the time when the sleep condition measuring function is activated, or measures the current time. The power supply unit 109 is, for example, a battery and supplies power to the other components of the sleep condition measuring apparatus 100 in accordance with a request from the control unit 110.

The control unit 110 controls the whole sleep condition measuring apparatus 100. Specifically, the control unit 110 transmits and receives various data to and from the other components and issues various process requests to the components.

The body motion data processing unit 120 acquires the user's body motion data via the control unit 110. The body motion data processing unit 120 carries out a process described below to derive a variation in body motion data and the amount of body motion which is the average value of a variation in body motion data. The body motion data is accelerations (G) in the directions of three axes as shown in FIG. 3A.

First, the body motion data processing unit 120 temporally differentiates the accelerations in the axial directions to obtain derivatives as shown in FIG. 3B. Then, the body motion data processing unit 120 calculates the square root of the square sum of the derivatives in the axial directions as shown in FIG. 3C. The body motion data processing unit 120 inputs the square root value to the control unit 110 as a variation in body motion data. Moreover, the body motion data processing unit 120 calculates the average value of the variation in body motion data every predetermined period (for example, one minute). The body motion data processing unit 120 then inputs the average value to the control unit 110 as the amount of body motion.

The pulse wave data processing unit 121 acquires the user's pulse wave data via the control unit 110, and carries out a process described below to acquire pulse wave interval data.

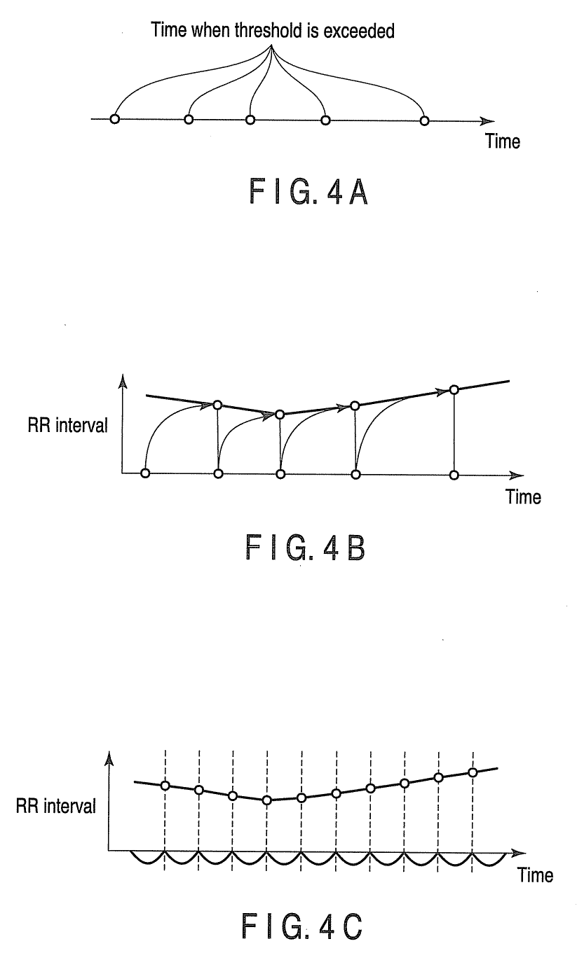

First, the pulse wave data processing unit 121 samples pulse wave data, and temporally differentiates the pulse wave sampling data to remove DC variation components. Then, for intervals each of about one second preceding and succeeding each sampling point in the pulse wave sampling data from which DC variation components has been removed, the pulse wave data processing unit 121 sets a predetermined value between a maximum value and a minimum value as a threshold for detection of a pulse wave interval. The pulse wave data processing unit 121, for example, sets the difference between the maximum value and the minimum value to be an amplitude and sets the threshold by adding 90% of such amplitude to the minimum value. The pulse wave data processing unit 121 determines the time when the pulse wave sampling data from which the DC variation components have been removed exceeds the threshold, as shown in FIG. 4A. Then, as shown in FIG. 4B, the pulse wave data processing unit 121 inputs the interval between the adjacent points in time to the control unit 110 as pulse wave interval (RR interval) data.

The pulse wave interval interpolating unit 122 acquires pulse wave interval data via the control unit 110 and interpolates the pulse wave interval data for re-sampling. The intervals among the respective pulse wave interval data derived by the pulse wave data processing unit 121, described above, correspond to the user's pulse wave intervals and are thus unequal. Thus, to allow the pulse wave interval analyzing unit 141, described below, to perform frequency analysis, the pulse wave interval interpolating unit 122 re-samples the interpolated data so that the pulse wave interval data has equal data intervals. Specifically, the pulse wave interval interpolating unit 122 generates, for example, a data set for one minute from the pulse wave interval data, and interpolates the pulse wave interval data for each data set using a high-order polynomial. For example, the pulse wave interval interpolating unit 122 performs three-order polynomial interpolation on the pulse wave interval data shown in FIG. 4B using three points, that is, each interpolation target point and points preceding and succeeding the interpolation target point. The pulse wave interval interpolating unit 122 then performs re-sampling to obtain interpolated pulse wave interval data at equal intervals such as the one shown in FIG. 4C. The pulse wave interval interpolating unit 122 inputs the interpolated pulse wave interval data to the control unit 110.

The fall-asleep/wake-up determining unit 130 acquires a variation in the user's body motion data via the control unit 110. The fall-asleep/wake-up determining unit 130 then determines the user's fall-asleep timing, wake-up timing, and nocturnal awakening timing. Here, the nocturnal awakening means arousal between the fall-asleep timing and the wake-up timing (the user's condition from the fall-asleep timing until the wake-up timing is hereinafter described as “asleep”) and includes instantaneous arousal. The fall-asleep/wake-up determining unit 130 includes a body motion determining unit 131 and an arousal determining unit 132.

The body motion determining unit 131 determines whether or not the user's body motion has occurred based on the user's body motion data. Specifically, the body motion determining unit 131 determines that the user's body motion has occurred when the variation in the body motion data is larger than a threshold for body motion determination (for example, 0.01 G/s).

The arousal determining unit 132 determines whether or not the user is awake based on the frequency of body motion determined by the body motion determining unit 131. Specifically, the arousal determining unit 132 determines that the user is awake if the number of body motions determined during a predetermined period (for example, one minute) is equal to or greater than a threshold (for example, five times/minute). On the other hand, the arousal determining unit 132 determines that the user is asleep if the number of body motions determined during the predetermined period is smaller than the threshold.

The fall-asleep/wake-up determining unit 130 determines the user's fall-asleep timing when the arousal determining unit 132 determines that the user is asleep, a predetermined number of consecutive times. The fall-asleep/wake-up determining unit 130 then records the fall-asleep timing in the recording unit 103 via the control unit 110. The fall-asleep/wake-up determining unit 130 determines the user's wake-up timing when the arousal determining unit 132 determines that the user is awake, a predetermined number of consecutive times. The fall-asleep/wake-up determining unit 130 then records the wake-up timing in the recording unit 103 via the control unit 110. If the arousal determining unit 132 determines that the user is awake while the user is in bed, the fall-asleep/wake-up determining unit 130 records the determination timing in the recording unit 103 as nocturnal awakening via the control unit 110.

The autonomic nerve index acquiring unit 140 acquires a sympathetic nerve index LF indicating the activity of the sympathetic nerves and a parasympathetic nerve index HF indicating the activity of the parasympathetic nerves based on the interpolated pulse wave interval data, as autonomic index indices indicating the conditions the user's autonomic nerves. The pulse wave synchronizes with the heartbeat. Thus, the indices indicating the condition of the autonomic nerves controlling the heartbeat are obtained based on the pulse wave interval of the user who is in bed. Specifically, the autonomic nerve index acquiring unit 140 acquires the interpolated pulse wave interval data in units of the data sets via the control unit 110. The autonomic nerve index acquiring unit 140 calculates the sympathetic nerve index LF and the parasympathetic nerve index HF based on frequency analysis results for the data set. The autonomic nerve index acquiring unit 140 records the sympathetic nerve index LF and the parasympathetic nerve index HF in the recording unit 103 via the control unit 110. Any of various methods such as an AR model, a maximum entropy method, and a wavelet method may be used as a frequency analyzing method. However, the autonomic nerve index acquiring unit 140 according to the present embodiment uses an FFT (Fast Fourier Transform) method, which imposes a reduced data processing load. The autonomic nerve index acquiring unit 140 includes a pulse wave interval analyzing unit 141 and an autonomic nerve index calculating unit 142.

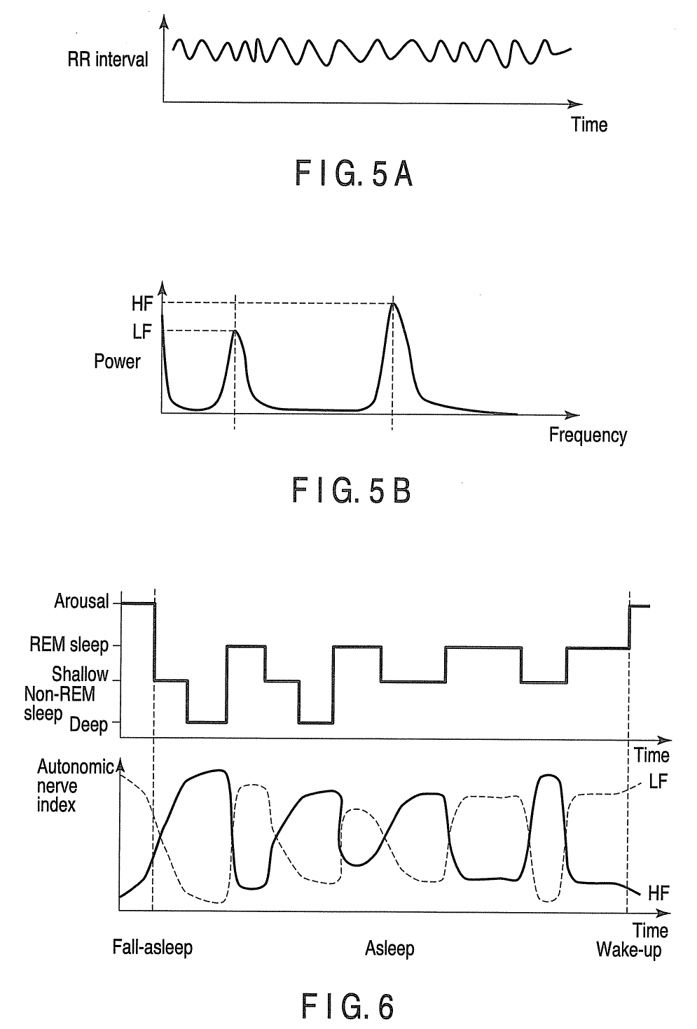

The pulse wave interval analyzing unit 141 performs FFT on such interpolated pulse wave interval data as shown in FIG. 5A in units of the data set to convert the interpolated pulse wave interval data into a frequency spectrum distribution.

The autonomic nerve index calculating unit 142 calculates a power spectrum distribution from the frequency spectrum distribution obtained by the pulse wave interval analyzing unit 141. For example, the autonomic nerve index calculating unit 142 calculates the power spectrum distribution shown in FIG. 5B from the frequency spectrum distribution in FIG. 5A, which relates to the interpolated pulse wave interval data. Then, the autonomic nerve index calculating unit 142 calculates each of the sympathetic nerve index LF and the parasympathetic nerve index HF based on a peak of a low frequency region (close to 0.05 to 0.15 Hz) and a peak of a high frequency region (close to 0.15 to 0.4 Hz) in the calculated power spectrum distribution. Specifically, the autonomic nerve index calculating unit 142 calculates the arithmetic average of power spectrum values for three points, that is, a data point indicating the peak of the low frequency region and data points preceding and succeeding the above-described data point to be the sympathetic nerve index LF. The autonomic nerve index calculating unit 142 calculates the arithmetic average of power spectrum values for three points, that is, a data point indicating the peak of the high frequency region and data points preceding and succeeding the above-described data point to be the parasympathetic nerve index HF.

Now, an example of a variation in the depth of sleep of a non-sleepless user during one night (sleep pattern) will be described. As shown in a lower stage of FIG. 6, while a normal adult is in bed, the sympathetic nerve index LF and the parasympathetic nerve index HF alternately become dominant. Although described below in detail, the depth of sleep of the user correspond approximately to the dominance level of the user's parasympathetic nerve index HF over the user's sympathetic nerve index LF. The depth of sleep shown in an upper stage of FIG. 6 is obtained based on the variations in the sympathetic nerve index LF and the parasympathetic nerve index HF, shown in the lower stage of FIG. 6. In the upper stage of FIG. 6, the user's depth of sleep varies as follows: “shallow sleep”→“deep sleep”→“REM sleep”→“shallow sleep”→“deep sleep”→“REM sleep”→“shallow sleep”→“REM sleep”→“shallow sleep”→+“REM sleep”. In this manner, a normal adult rhythmically repeats a relatively shallow sleep and a relatively deep sleep according to given units (sleep cycles). Each of the sleep cycles of a normal adult is known to last about 90 minutes. Now, attention is focused on each sleep cycle. In a relatively initial sleep cycle, the “deep sleep” appears during many periods. In a relatively terminal sleep cycle, the “REM sleep” appears during many periods. That is, the comprehensive tendency of a normal adult's sleep is such that the user's depth of sleep changes from deep sleep to shallow sleep as time elapses from the fall-asleep timing.

The parasympathetic nerve dominance level calculating unit 150 calculates the parasympathetic nerve dominance level based on the user's sympathetic nerve index LF and parasympathetic nerve index HF via the control unit 110. Specifically, the parasympathetic nerve dominance level calculating unit 150 acquires the user's sympathetic nerve index LF and parasympathetic nerve index HF via the control unit 110 to calculate the parasympathetic nerve dominance level. The parasympathetic nerve dominance level calculating unit 150 then records the parasympathetic nerve dominance level and the time elapsed from the fall-asleep timing as corresponding temporal information in the recording unit 103 via the control unit 110. The parasympathetic nerve dominance level calculating unit 150 includes a comparison unit 151.

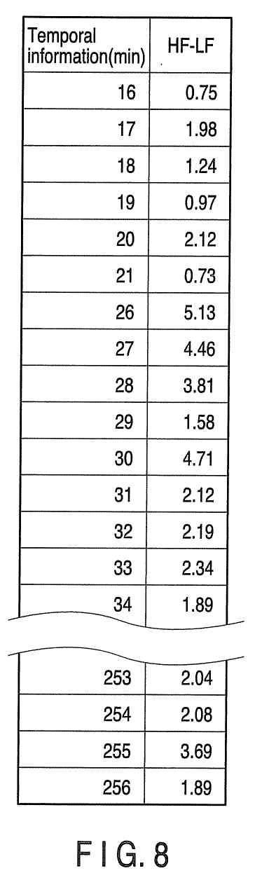

The comparison unit 151 compares the parasympathetic nerve index HF with the sympathetic nerve index LF in terms of magnitude correlation. If the parasympathetic nerve index HF is equal to or greater than the sympathetic nerve index LF, the comparison unit 151 calculates HF−LF as a parasympathetic nerve dominance level. The calculated parasympathetic nerve dominance level HF−LF is labeled based on the time elapsed from the fall-asleep timing as corresponding temporal information. The labeled, calculated parasympathetic nerve dominance level HF−LF is recorded in the recording unit 103 via the control unit 110. For example, as shown in FIG. 8, the temporal information and the parasympathetic nerve dominance level are recorded in the recording unit 103 in association with each other. Furthermore, a difference normalizing unit 152 that divides HF−LF, described above, by HF for normalization may be provided in the parasympathetic nerve dominance level calculating unit 150. In this case, the result of the normalization (HF−LF)/HF may be determined to be a parasympathetic nerve dominance level.

The comfort level acquiring unit 160 acquires the comfort level as an index indicating the user' sleep condition, and records the comfort level in the recording unit 103 via the control unit 110. Here, the comfort level is an index indicating the quality of the user's sleep. Specifically, the comfort level acquiring unit 160 acquires the parasympathetic nerve dominance level and the corresponding temporal information via the control unit 110. The comfort level acquiring unit 160 then uses a weight the value of which is determined by the temporal information, to calculate the comfort level. The comfort level acquiring unit 160 includes a weight calculating unit 161 and a comfort level calculating unit 162.

The weight calculating unit 161 calculates the weight according to a function decreasing as the time elapsed from the fall-asleep timing. Since the temporal information is the time elapsed from the fall-asleep timing as described above, the weight calculating unit 161 calculates the weight using the temporal information as an input to the function. For example, the decreasing function decreases monotonously as the time elapses from the fall-asleep timing.

The fall-asleep timing is defined as 0, the wake-up timing is defined as T, and the time (min) elapsed from the fall-asleep timing is defined as t. A specific example of a weight W(t) corresponding to the elapsed time t will be described. For example, the weight W(t) may be linear (a linear expression of t) as in the case of W(t)=1−t/T or inversely proportional to the elapsed time t as in the case of W(t)=1/t, or may decease according to a LOG scale as in the case of W(t)=1/log(t+1). More preferably, the weight W(t) may be determined in view of the fact that the normal user's sleep rhythm is such that one cycle lasts 90 minutes. Specifically, the weight W(t) may utilize a sigmoid function such as W(t)=1/(1+EXP((t−90*N)/10)) or may be stepped as in the case of W(t)=1(t<90*N) and W(t)=0(90*N≦t) (N is desirably 1 or 2). Furthermore, if W(t) is a stepped weight, W(t) is not limited to the above-described binary weight but may be a multivalued weight that decreases every 90 minutes. Owing to individual differences in sleep cycles, the value of 90 minutes may be replaced with another one such as an average value obtained by measuring the user's recent sleep cycles.

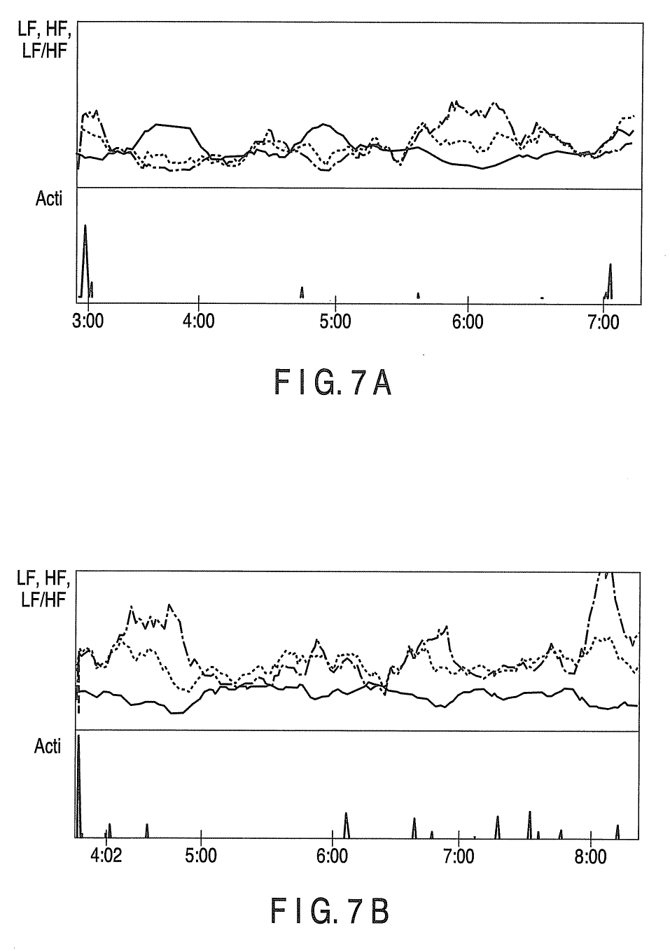

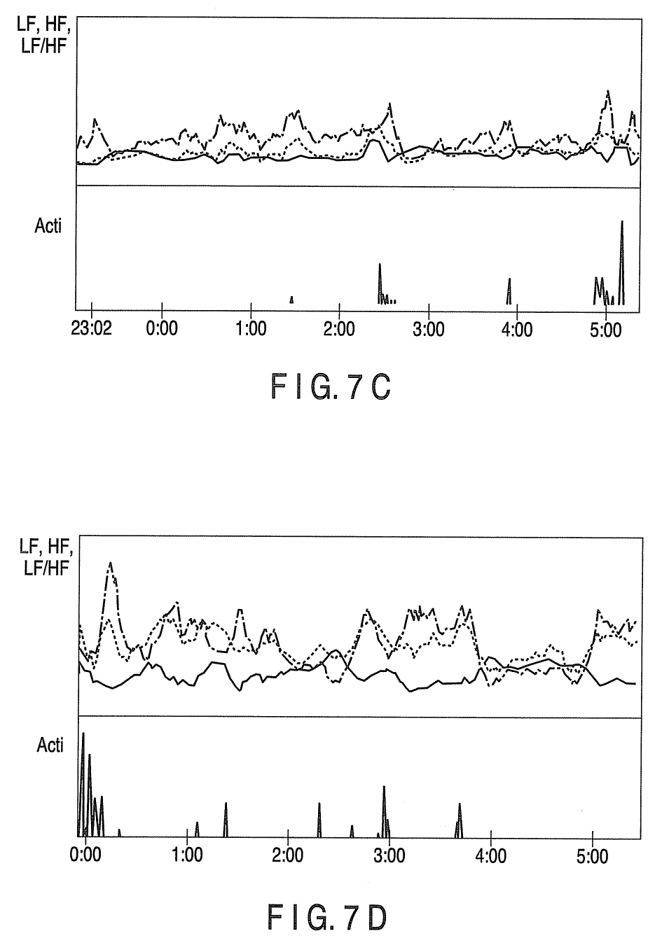

With reference to FIGS. 7A, 7B, 7C, and 7D, description will be given of the technical significance of the use, for calculation of the comfort level, of the weight decreasing as the time elapsed from the fall-asleep timing. FIGS. 7A, 7B, 7C, and 7D show the results of calculation of the autonomic nerve indices for four adult users measured during one night. In FIGS. 7A, 7B, 7C, and 7D, the axis of abscissa indicates the actual time, and the axis of ordinate indicates the autonomic nerve indices HF (dotted line) and LF (solid line) and LF/HF (dashed line) in an upper stage, and the amount of body motion Acti in a lower stage.

A user A corresponding to FIG. 7A is not sleepless and exhibits Pittsburgh sleep quality index (PSQI) of 4; PSQI indicates the user's subjective quality of sleep (a person with PSQI of at least six is defined to be sleepless, and a user with PSQI of less than six is defined to be non-sleepless). As shown in FIG. 7A, while the user A is in bed, the parasympathetic nerves are rhythmically dominant over the sympathetic nerves. The duration for which the parasympathetic nerves are dominant decreases as the time elapses. That is, as is the case with the normal sleep patterns described above, the user A's sleep is such that a change between a shallow sleep and a deep sleep is rhythmically repeated as sleep cycles, and in a broad sense, the depth of sleep decrease as the time elapses from the fall-asleep timing.

A user B corresponding to FIG. 7B is sleepless and exhibits PSQI of 14. As shown in FIG. 7B, while a user B is in bed, there appears, during the night, no period during which the parasympathetic nerves are dominant over the sympathetic nerves, and the sympathetic nerves are constantly dominant.

A user C corresponding to FIG. 7C is sleepless and exhibits PSQI of 10. As shown in FIG. 7C, the user C's sleep is such that the activity level of the parasympathetic nerves is close to that of the sympathetic nerves, and a number of intersections of them are shown. However, the user C's sleep lacks a rhythm like that of the user A's sleep and deviates from the normal sleep pattern. Furthermore, the duration for which the parasympathetic nerves are dominant is short, and the dominance level of the parasympathetic nerves is low. However, since such a period appears frequently, the total length of the periods during one night is relatively large. Furthermore, for the user A, the number of the periods during which the parasympathetic nerves are dominant decreases as the time elapses from the fall-asleep timing. However, for the user C, such a tendency is not exhibited, and the total length of the periods is expected to increase consistently with the duration for which the user C stays in bed.

A user D corresponding to FIG. 7D is sleepless and exhibits PSQI of 12. As shown in FIG. 7D, in contrast to the user A's sleep, the user D's sleep is such that a shallow sleep appears during an initial period, whereas a deep sleep appears during a terminal period. That is, the length of the period during which the parasympathetic nerves are dominant increases towards the end of the sleep duration. This also deviates from the normal sleep pattern. The user D is also aware of the user D's own sleeplessness.

The comfort level is an index indicating the quality of sleep. Thus, desirably, a relatively high comfort level is calculated for the user A, while relatively low comfort levels are calculated for the users B, C, and D. As described above, the conventional technique (JP-A 2007-130181) calculates the comfort level simply by temporally integrating the parasympathetic nerve dominance level. When the comfort level is calculated for the users A and B according to the conventional technique, since the user B's sleep does not involve the period during which the parasympathetic nerves are dominant, a comfort level lower than that for the user A is calculated for the user B. However, when the comfort level is calculated for the users C and D according to the conventional technique, since the sleeps of the users C and D deviate from the normal sleep pattern but involve the period during which the parasympathetic nerves are dominant, the comfort level is not necessarily low for the users C and D.

As described above, sleepless persons exhibit various sleep patterns. Thus, the comfort level needs to be determined taking the normal human sleep pattern into account rather than being determined simply by integrating the parasympathetic nerve dominance level. That is, in view of the fact that the depth of human sleep generally decreases as the time elapses from the fall-asleep timing, high comfort levels need to be prevented from being calculated for a non-rhythmical sleep pattern like the user C's and a sleep pattern such as the user D's which opposes the normal sleep pattern. Thus, the comfort level acquiring unit 160 according to the present embodiment uses the weight decreasing as the time elapses from the fall-asleep timing, to calculate the comfort level with higher importance placed on the parasympathetic nerve dominance level during the initial period of sleep than on the parasympathetic nerve dominance level during the terminal period of sleep. The use of such a weight allows a low comfort level to be calculated for a sleep pattern opposing the normal sleep pattern, for example, the user D's sleep pattern.

The comfort level calculating unit 162 multiplies each of the parasympathetic nerve dominance levels acquired via the control unit 110, by a corresponding weight. Here, the corresponding weight is calculated, by the weight calculating unit 161, for the temporal information corresponding to each parasympathetic nerve dominance level. That is, the parasympathetic nerve dominance level is multiplied by the weight decreasing with respect to the elapsed time indicated by the corresponding temporal information. Then, the comfort level calculating unit 162 integrates each of the weighted parasympathetic nerve dominance levels from the time when the user falls asleep until the user wakes up. The comfort level calculating unit 162 then records an integration result in the recording unit 103 via the control unit 110 as a comfort level. Alternatively, the comfort level calculating unit 162 may record the maximum value of the weighted parasympathetic nerve dominance level in the recording unit 103 via the control unit 110 as a comfort level.

The parasympathetic nerve dominance levels used by the comfort level calculating unit 162 to calculate the comfort level may be limited to those higher than a predetermined threshold. Neglecting relatively low parasympathetic nerve dominance levels allows a relatively low comfort level to be calculated for a sleep pattern such as the user C's in which the activity level of the parasympathetic nerves is close to that of the sympathetic nerves.

The easiness-of-falling-asleep calculating unit 171 calculates the user's easiness-of-falling-asleep as an index indicating the user's sleep condition, and inputs the user's easiness-of-falling-asleep to the control unit 110. Here, the user's easiness-of-falling-asleep is the reciprocal of sleep latency (a duration from a go-to-bed timing until the fall-asleep timing). The go-to-bed timing may be, for example, the time when the user provides the input unit 101 with a user input instructing the control unit 110 to activate the sleep condition measuring function or the time when the user provides the input unit 101 with an input indicating that the user has gone to bed. Alternatively, the angle of the user's wrist may be calculated from the accelerations in the directions of the three axes for the user which are obtained by the acceleration sensor 104 so that the go-to-bed timing can be determined based on the angle of the wrist. Specifically, the go-to-bed timing may be determined when the frequency at which the angle of the wrist is close to the angle of the arm in the supine position is greater than a predetermined value. On the other hand, the fall-asleep timing is determined by the fall-asleep/wake-up determining unit 130 and recorded in the recording unit 103 via the control unit 110.

The sleep depth determining unit 172 determines the user's depth of sleep as an index indicating the user's sleep condition, and inputs the depth of sleep to the control unit 110. The sleep depth determining unit 172 acquires the sympathetic nerve index LF and the parasympathetic nerve index HF via the control unit 110 to determine the user's depth of sleep at the time of calculation of LF and HF. Specifically, the sleep depth determining unit 172 determines that the user is in a deep sleep when the value of LF/HF is smaller than a first threshold and the value of HF is greater than a second threshold. The sleep depth determining unit 172 determines that the user is in a REM sleep when the value of LF/HF is greater than a third threshold (which is greater than the first threshold), the value of HF is smaller than a fourth threshold (which is smaller than the second threshold), and the sum of standard deviations of LF and HF is greater than a fifth threshold. The sleep depth determining unit 172 determines that the user is in a shallow sleep when neither the determination conditions for the deep sleep nor the determination conditions for the REM sleep are met.

The average body motion amount calculating unit 173 calculates the user's average body motion amount as an index indicating the user's sleep condition, and inputs the user's average body motion amount to the control unit 110. Specifically, the average body motion amount calculating unit 173 acquires the body motion amount via the control unit 110 to calculate the user's average body motion amount during sleep.

The nocturnal awakening acquiring unit 174 acquires the number of the user's nocturnal awakenings and the total time of the nocturnal awakenings as indices indicating the user's sleep condition, and inputs these indices to the control unit 110. Specifically, the nocturnal awakening acquiring unit 174 acquires the user's nocturnal awakening timing via the control unit 110 to calculate the number of nocturnal awakenings and the total time of the nocturnal awakenings. If the nocturnal awakening timings are temporally consecutive, the nocturnal awakening acquiring unit 174 considers the series of awakening timings to be one nocturnal awakening. The nocturnal awakening acquiring unit 174 may acquire either one of the number and total time of the user's nocturnal awakenings.

The statistical processing unit 175 acquires various indices such as the comfort level, the easiness-of-falling-asleep, the depth of sleep, the average body motion amount, and the nocturnal awakening from the control unit 110. The statistical processing unit 175 then statistically processes the indices and returns processing results to the control unit 110. Specifically, the statistical processing unit 175 calculates an average value and a standard deviation for each of the indices for a plurality of nights. The statistical processing unit 175 also calculates T-scores based on indices obtained from other users. For the body motion and the nocturnal awakening, a smaller value indicates a better sleep condition. Thus, in this case, the T-score is desirably calculated after calculating the reciprocal. Thus, when the indices are displayed in T-score form by the display unit 102, the user can more easily determine the user's own sleep condition.

As described above, the sleep condition measuring apparatus uses the weight decreasing as the time elapses from the fall-asleep timing, to calculate the comfort level as an index indicating the quality of sleep. Therefore, the sleep condition measuring apparatus according to the present invention enables the comfort level to be calculated taking the normal adult's sleep pattern into account. A more reliable comfort level can thus be obtained.

### Second Embodiment

A sleep condition measuring apparatus according to a second embodiment of the present invention corresponds to the sleep condition measuring apparatus shown in FIG. 1 and in which the comfort level calculating unit 162 is replaced with a comfort level calculating unit 262. In the description below, the same components as those in FIG. 1 are denoted by the same reference numerals. Differences from the first embodiment will be mainly described below.

The comfort level calculating unit 262 calculates the length of the period (that is, the duration) during which the parasympathetic nerves are dominant, based on temporal information corresponding to the parasympathetic nerve dominance level acquired via the control unit 110. If the parasympathetic nerve dominance level and temporal information shown in FIG. 8 are taken as an example, the parasympathetic nerves are dominant during a period from 16 [min] to 21 [min]. The comfort level calculating unit 262 calculates the duration of 6 [min].

Unlike the above-described comfort level calculating unit 162, the comfort level calculating unit 262 uses the duration of the period during which the parasympathetic nerves are dominant, instead of the parasympathetic nerve dominance level, to calculate the comfort level. Specifically, the comfort level calculating unit 262 multiplies the duration (in the above-described example, 6 [min]) by a weight corresponding to the start point (in the above-described example, 16 [min]) of the period. The weight by which the duration is multiplied is not limited to the above-described one but for example, may correspond to the end point (in the above-described example, 21 [min]) of the period or the center (in the above-described example, 19 [min]) of the period, or may be the average value of the weights corresponding to all the pieces of temporal information included in the period. Then, the comfort level calculating unit 262 integrates the weighted durations from the time when the user falls asleep until the user wakes up. The comfort level calculating unit 262 records a calculation result in the recording unit 103 as a comfort level. Alternatively, the comfort level calculating unit 162 may record the maximum value of the weighted duration in the recording unit 103 via the control unit 110 as a comfort level.

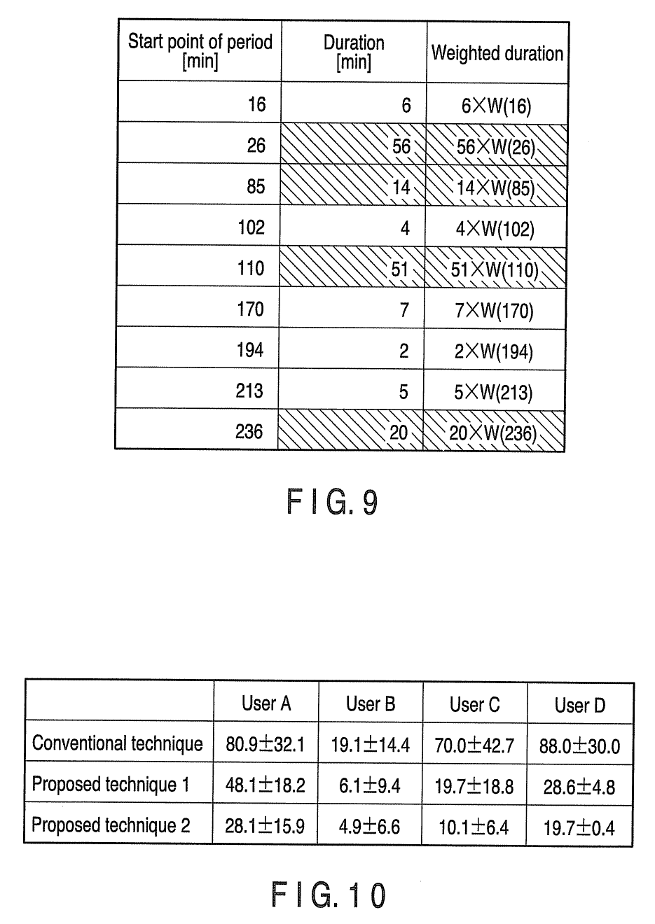

The parasympathetic nerve dominance level used by the comfort level calculating unit 262 to calculate the duration may be limited to one that is equal to or greater than a predetermined threshold. Neglecting relatively low parasympathetic nerve dominance levels allows a relatively low comfort level to be calculated for a sleep pattern such as the user C's in which the activity level of the parasympathetic nerves is close to that of the sympathetic nerves. Alternatively, the duration used by the comfort level calculating unit 262 may be limited to one that is equal to or greater than a predetermined threshold (for example, 10 minutes). Specifically, as shown in FIG. 9, if the duration for which the parasympathetic nerves are dominant is at least 10 minutes (this is shown by a shaded portions), the comfort level calculating unit 262 may multiply the duration by a weight W(t) corresponding to the start point of the period and use the weighted duration to calculate the comfort level. Neglecting periods with relatively short durations allows a relatively low comfort level to be calculated for a sleep pattern such as the user C's in which the activity level of the parasympathetic nerves is close to that of the sympathetic nerves.

The effects of the sleep condition measuring apparatus according to the present embodiment will be described with reference to FIG. 10. FIG. 10 shows average values and standard deviations obtained by calculating the comfort level for the above-described users A, B, C, and D for a plurality of nights according to the conventional technique and proposed techniques 1 and 2. The conventional technique calculates the comfort level simply by integrating the parasympathetic nerve dominance levels obtained while the user is asleep (without weighting the parasympathetic nerve dominance levels). The proposed techniques 1 and 2 uses a weighting function W(t)=1/(1+EXP((t−90)/10)) and places high importance on the depth of sleep (parasympathetic nerve dominance level) during the user's first sleep cycle. More specifically, according to the proposed technique 1, if the duration of the period during which the parasympathetic nerves are dominant is at least 10 minutes, the duration is multiplied by the weight (t) corresponding to the start point of the period to calculate the integrated value of the weighted durations as a comfort level. According to the proposed technique 2, if the duration of the period during which the parasympathetic nerves are dominant is at least 10 minutes, the duration is multiplied by the weight (t) corresponding to the start point of the period to calculate the maximum value of the weighted durations as a comfort level.

As shown in FIG. 10, any of the conventional technique and the proposed techniques 1 and 2 calculates a lower comfort level for the user B than for the user A. On the other hand, with the conventional technique, the comfort levels calculated for the users C and D is equivalent to or higher than that calculated for the user A. Thus, the conventional technique fails to reflect the actual quality of sleep of the users C and D, who are sleepless. In contrast, according to the proposed techniques 1 and 2, the comfort levels calculated for the users C and D are sufficiently lower than that calculated for the user A. Therefore, the proposed techniques 1 and 2 are expected to offer more reliable comfort levels than the conventional technique.

As described above, the sleep condition measuring apparatus uses the weight decreasing as the time elapses from the fall-asleep timing to calculate the comfort level as an index indicating the quality of sleep. Therefore, the sleep condition measuring apparatus according to the present invention enables the comfort level to be calculated taking the general adult's sleep pattern into account. A more reliable comfort level can thus be obtained.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A sleep condition measuring apparatus comprising:
a first detection unit configured to detect body motion of a user;
a determination unit configured to determine a first timing indicating that the user has fallen asleep and a second timing indicating that the user has waken up, based on the body motion;
a second detection unit configured to detect a pulse wave interval of the user;
an acquisition unit configured to acquire a first index indicating activity of a sympathetic nerve of the user and a second index indicating activity of a parasympathetic nerve of the user, based on the pulse wave interval;
a first calculation unit configured to calculate a dominance level of the second index over the first index every predetermined time; and
a second calculation unit configured to calculate a third index indicating quality of sleep of the user from the first timing until the second timing, using the dominance level and a weight decreasing as time elapses from the first timing.

2. The apparatus according to claim 1, wherein the second calculation unit weights the dominance level using the weight, and integrates the weighted dominance level from the first timing until the second timing to calculate the third index.

3. The apparatus according to claim 1, wherein if the dominance level is equal to or greater than a threshold, the second calculation unit weights the dominance level using the weight, and integrates the weighted dominance level from the first timing until the second timing to calculate the third index.

4. The apparatus according to claim 1, wherein the second calculation unit weights the dominance level using the weight, and calculates, as the third index, a maximum value of the weighted dominance level obtained from the first timing until the second timing.

5. The apparatus according to claim 1, wherein the second calculation unit calculates a duration for which the second index is dominant over the first index, based on time of calculation of the dominance level by the first calculation unit, then weights the duration using a weight corresponding to a start point of the duration, and integrates the weighted duration from the first timing until the second timing to calculate the third index.

6. The apparatus according to claim 1, wherein the second calculation unit calculates a duration for which the dominance level is equal to or greater than the threshold, the duration is based on time of calculation of the dominance level by the first calculation unit, then weights the duration using the weight corresponding to the start point of the duration, and integrates the weighted duration from the first timing until the second timing to calculate the third index.

7. The apparatus according to claim 1, wherein the second calculation unit calculates a duration for which the second index is dominant over the first index, based on the time of the calculation, then weights the duration using the weight corresponding to the start point of the duration, and calculates a maximum value of the weighted duration obtained from the first timing until the second timing, as the third index.

8. The apparatus according to claim 1, wherein the weight has a first value when the elapsed time is within a first sleep cycle of the user, and has a second value smaller than the first value when the elapsed time surpasses the first sleep cycle.

9. The apparatus according to claim 8, wherein the sleep cycle lasts 90 minutes.

10. A sleep condition measuring method comprising:
detecting body motion of a user;
determining a first timing indicating that the user has fallen asleep and a second timing indicating that the user has waken up, based on the body motion;
detecting a pulse wave interval of the user;
acquiring a first index indicating activity of a sympathetic nerve of the user and a second index indicating activity of a parasympathetic nerve of the user, based on the pulse wave interval;
calculating a dominance level of the second index over the first index every predetermined time; and
calculating a third index indicating quality of sleep of the user from the first timing until the second timing, using the dominance level and a weight decreasing as time elapses from the first timing.
